(19) United States
(12) Patent Application Publication
(10) Pub. No.: US 2025/0195099 A1
(43) Pub. Date: Jun. 19, 2025

(54) INNER TUBE FOR A SURGICAL CUTTING DEVICE AND METHOD OF MANUFACTURING THE SAME

(71) Applicant: SWISS MEDICAL INSTRUMENTS AG, Wollerau (CH)

(72) Inventor: André Gasser, Wollerau (CH)

(73) Assignee: SWISS MEDICAL INSTRUMENTS AG, Wollerau (CH)

(21) Appl. No.: 18/844,931

(22) PCT Filed: Mar. 8, 2023
(86) PCT No.: PCT/IB2023/052219

§ 371 Date: Sep. 6, 2024

(30) Foreign Application Priority Data
Mar. 8, 2022 (CH) CH000246/2022

(51) Int. Cl. A61B 17/32; A61B 17/00

(52) U.S. Cl. CPC A61B 17/32002; A61B 2017/00526

(57) ABSTRACT

The invention relates to a method of manufacturing an inner tube for a surgical cutting device comprising
a. an outer tube having an outer tube head portion at a distal end of the outer tube;
b. said outer tube head portion having an outer tube window; wherein
c. the inner tube can be rotatably disposed in the outer tube;
d. an inner tube head portion is provided at a distal end of the inner tube; wherein
e. at least one cutting edge is formed on the inner tube head portion, which cutting edge is moved out of an interior of the outer tube when the inner tube is rotated through the outer tube window and is moved back into the interior of the outer tube when the inner tube is further rotated through the outer tube window; the method comprising the following steps:
f. providing an inner tube middle portion having a distal end;
g. forming the inner tube head portion at the distal end of the inner tube middle portion by an additive manufacturing process to obtain the inner tube.

The invention relates to the field of surgical cutting devices. In particular, it relates to an inner tube for a surgical cutting device and a method for its manufacture according to the independent patent claims.

In closed or endoscopic surgery, among other things, surgical cutting devices are used which comprise two telescoped tubes with their longitudinal axes parallel to each other, on which a so-called “cutting head” is located at one distal end and a coupling for connection, for example to a drive turbine, is located at the other end. End portions at the distal end may be open or closed at the front and also have forward-facing lateral openings through which an irrigation fluid may be supplied and tissue removed during a corresponding surgical procedure, in particular suctioned off, in particular together with the irrigation fluid. Said tubes generally have a small diameter d in the range of a few millimeters, in particular with 2.5 mm<d<10 mm, and a comparatively large length l, in particular in a range of 15 cm<l<30 cm.

Exemplary surgical cutting devices of the type mentioned are described in EP 1773215 A1, the contents of which are to be incorporated by reference in their entirety.

Said cutting instruments comprise an inner tube and an outer tube as two telescoped tubes, the inner tube being rotatable about the longitudinal axis thereof and/or relative to the outer tube, in particular by means of the drive turbine.

An outer tube head portion is provided at a distal end of the outer tube, which is also referred to as the outer head portion (in particular in EP 1773215 A1). An inner tube head portion is provided at a distal end of the inner tube, which (in particular in EP 1773215 A1) is also referred to as the inner head portion. The outer tube head portion and/or inner tube head portion may each have, in particular in the region of their respective distal ends, one or more cutting edges which slide and/or shear past one another when the inner tube rotates about its longitudinal axis and/or relative to the outer tube, and may thus cut off tissue, bones and/or other body components in a manner similar to scissors, in particular relatively soft tissue such as connective tissue, muscles, cartilage or tendons. To cut off such tissue, two cutting edges are therefore generally required to work together, a first of which is formed as part of the outer tube, in particular as part of the outer tube head portion, and a second of which is formed as part of the inner tube, in particular as part of the inner tube head portion.

A cutting edge formed on the inner tube is also referred to as the inner cutting edge; a cutting edge formed on the outer tube is referred to as the outer cutting edge.

In order to ensure sufficient mechanical stability of the inner tube, the inner tube, in particular the inner tube head portion, is usually at least partially closed at its distal end. The cutting edges are usually provided at one or more so-called inner openings, which are also referred to below as cutting windows. A cutting window may thereby be formed by an at least essentially lateral opening in the inner tube, wherein the cutting edges of a cutting window are formed in particular in an area of the remaining material of the inner tube surrounding the cutting window, in particular of the inner tube head portion, which delimit the cutting window in a circumferential direction of the inner tube.

Through an opening provided in a distal area, in particular at the distal end, of the outer tube, which is also referred to as the outer tube window, the inner cutting edge may be moved out of an interior of the outer tube through the outer tube window by rotating the inner tube and, upon further rotation, may be moved back into the interior of the outer tube through the outer tube window. The inner cutting edge may slide or shear past the outer cutting edge, which is preferably formed at the edge of the outer tube window, and cut off tissue in the process. In particular, the outer tube window may be directed laterally towards the front.

Cutting windows and/or outer tube windows are usually formed by means of material removal, in particular by means of an erosion process, in particular by means of wire erosion, in or from a precursor of the inner tube, in particular the inner tube head portion, in particular simultaneously with any cutting edges to be provided.

However, such erosion processes impose restrictions on the geometries that can be realized. This applies in particular to cutting windows provided or to be provided on the inner tube, where machining on the inside of a wall forming the inner tube is generally required to form optimum cutting edges. In the case of wire erosion processes, such restrictions result in particular from the fact that a respective working area of the erosion process must be accessible from two opposite sides in order to be able to guide a straight-tensioned erosion wire to and from the working point. The working area of the erosion process may be understood as the shortest distance between the erosion wire and the workpiece. In particular, this limits the minimum cutting angles that can be realized for the internal cutting edges, which may be defined as the angle β of the internal cutting edge to a tangent to the outside in analogy to EP 1773215 A1. In practice, no cutting angles of less than 30° can be produced, i.e. β>30° must apply.

Although so-called sinking, drilling or bar erosion processes allow greater degrees of freedom, as the working area only needs to be accessible from one side, only larger radii of curvature can be realized due to the larger cross-section and/or diameter of the erosion electrodes required to achieve sufficient mechanical stability compared to the erosion wire, which can be particularly obstructive when forming the cutting edges and can entail other restrictions.

In order to mitigate the limitations described above with regard to possible geometries in wire EDM, the inner tube head portion is often produced from a separate precursor or blank in the form of a relatively short portion of tube, whereby the EDM wire can be fed through an open, in particular proximal, end of the portion of tube. This may be necessary in particular for the production of an inner tube head portion with a single cutting window; however, it entails massive restrictions with regard to possible cutting window geometries. In addition and/or alternatively, several cutting windows may be provided at the same time, which are offset in relation to each other with respect to the circumferential direction, in particular opposite each other, whereby the erosion wire is guided through two cutting windows at the same time. However, such a procedure in turn entails considerable restrictions with regard to possible cutting window and edge geometries, particularly in the case of an odd number of cutting windows.

In addition, the separately produced inner tube head portion must then be connected and/or attached to an inner tube middle portion, for example by welding, in order to obtain a complete inner tube. However, this may cause several potential problems: Firstly, an inner diameter or inner cross-section of an inner pipe produced in this way may be reduced in an area of a resulting welded joint. This may lead to restrictions and/or obstructions in the removal, in particular suction, of flushing fluid, especially if it carries a large amount of tissue with it.

Unevenness of the welded joint on the outside of the inner tube may also hinder the rotation of the inner tube relative to the outer tube.

Finally, welding requires a further work step that complicates, increases the cost and/or slows down the production of the inner pipe.

To remove harder tissue, such as bone in particular, the inner tube head portion may also be designed as a milling head in the area of the distal end. In particular, a milling head may have at least one cutting edge which may also remove material, in particular harder material, without interacting with another cutting edge. A correspondingly designed inner tube may be inserted into an outer tube of the same or similar design instead of the inner tube with one or more cutting windows described above. The rotating milling head then protrudes from the outer tube in the area of the outer tube window and may remove hard tissue in this area in a controlled manner.

The invention is therefore based on the task of providing a method of manufacturing a surgical cutting device and a surgical cutting device obtainable by this method which avoids the above-mentioned disadvantages.

### BRIEF DESCRIPTION OF THE INVENTION

According to the invention, this task is solved by a method for manufacturing an inner tube for a surgical cutting device and by a surgical cutting device with an inner tube which may be manufactured by the method according to the independent patent claims.

A method of manufacturing an inner tube for a surgical cutting device comprising
a. an outer tube having an outer tube head portion at a distal end of said outer tube;
b. said outer tube head portion having an outer tube window; wherein
c. the inner tube can be rotatably disposed in the outer tube;
d. an inner tube head portion is provided at a distal end of the inner tube; wherein
e. at least one cutting edge is formed on the inner tube head portion, which cutting edge is moved out of an interior of the outer tube through the outer tube window when the inner tube is rotated and is moved back into the interior of the outer tube through the outer tube window when the inner tube is further rotated; may comprise the following method steps:
f. providing an inner tube middle portion having a distal end;
g. forming the inner tube head portion at the distal end of the inner tube middle portion using an additive manufacturing process to obtain the inner tube.

A method of manufacturing a surgical cutting device comprising
a. an outer tube having an outer tube head portion at a distal end of the outer tube;
b. an inner tube rotatably disposed in the outer tube with an inner tube head portion at a distal end of said inner tube; wherein
c. the outer tube head portion has an outer tube window;
d. at least one cutting edge is formed on the inner tube head portion, which cutting edge is moved out of an interior of the outer tube through the outer tube window when the inner tube is rotated and is moved back into the interior of the outer tube through the outer tube window when the inner tube is further rotated; may comprise the following method steps:
e. providing an inner tube middle portion having a distal end;
f. forming the inner tube head portion at the distal end of the inner tube middle portion using an additive manufacturing process to obtain the inner tube;
g. inserting the inner tube into the outer tube.

The methods according to the invention described above are based on the idea of forming the inner tube head portion at a distal end of an inner tube middle portion by means of an additive manufacturing process, in particular a powder bed-based additive manufacturing process, in one operation, in particular directly and/or immediately at said distal end, simultaneously connecting it to the inner tube middle portion and providing all geometric and/or structural features. In a sense, the inner tube middle portion serves as a blank and/or precursor for the inner tube.

A straight, in particular cylindrical, tube middle portion, in particular with a length between 10 cm and 20 cm, may be selected as the inner tube middle portion, preferably made of a material suitable and/or approved for surgical applications, in particular stainless steel. The inner tube middle portion may comprise a portion designed as a flexible shaft. An inner and/or outer cross-section of the inner tube middle portion may be homogeneous over the entire length.

The inner tube middle portion may be positioned inside a working volume of a device for performing an additive manufacturing process, in particular inside a 3D printing chamber, in such a way that a longitudinal axis of the inner tube middle portion is aligned at least substantially vertically, i.e. in a vertical direction. Preferably, a plurality of inner tube middle portions, in particular between 50 and 500 inner tube middle portions, may be positioned next to each other and/or parallel to each other. The inner tube middle portions may be arranged in two horizontal directions perpendicular to the vertical direction in such a way that they utilize the working volume as well as possible.

The working volume may then be filled with a powder that is necessary and suitable for carrying out the additive manufacturing process to such an extent that the one or more inner tube middle portions are completely surrounded by the powder, so that in particular the distal end of the inner tube middle portions does not protrude from the powder.

By means of the additive manufacturing process, an inner tube head portion may then be formed layer by layer on the distal ends of the one or more inner tube middle portions, which in particular may be at least approximately circular, whereby a short tubular region may be formed first to increase mechanical stability, which preferably has an identical inner and/or outer cross-section and/or diameter as the one or more inner tube middle portions, and preferably a length of a few millimeters to a few centimeters.

Adjacent to the short tubular region, which may form a proximal end of the inner tube head portion, a cutting window region may be formed, which comprises one or more cutting windows. Several cutting windows may have an identical shape, at least in pairs, and/or be evenly distributed around the cutting window area with respect to a circumferential direction.

Using the additive manufacturing process, any number of small cutting angles may be formed for each cutting edge. In particular, an angle β of the inner cutting edge to a tangent to the outside of β<10° can be realized, especially β<8°, as is common with razor blades, for example.

The additive manufacturing process makes it possible to provide at least approximately any shape, in particular three-dimensional shapes, both on an inner side and on an outer side of an inner tube head portion, which allows unlimited shaping, in particular the provision of free surfaces and/or free shapes both on the inner side and on the outer side. Conventional manufacturing processes, such as erosion in particular, do not allow such unlimited shaping, so that certain shapes cannot be produced with the latter processes. However, even shapes that can in principle be realized by means of eroding may be excluded from practical usability, as production may be too time-consuming and/or resource-intensive—for example, it may require the use of 5-axis processing machines, in particular 5-axis grinding machines, on which only a single inner tube middle portion may be processed at any one time.

The additive manufacturing process makes it possible, for example, to design the short tubular area in such a way that an inner diameter and/or cross-section tapers and widens again several times, and in particular has a regular, preferably sinusoidal shape. Surprisingly, it has been found that such a course may avoid or prevent obstructions during the removal, in particular suction, of irrigation fluid, especially if this carries a large amount of tissue with it.

Adjacent to the cutting window area, an inner tube head portion may be formed, which at least partially closes the inner tube head portion at its distal end and/or forms a distal end of the inner tube head portion.

Instead of an inner tube head portion with one or more cutting windows as described above, an inner tube head portion may also be produced at the distal end of which a milling head is formed.

The powder may be selected such that the one or more inner tube head portions metallurgically correspond as closely as possible to the one or more inner tube middle portions, in particular form an alloy with a composition that corresponds as closely as possible to the one or more inner tube middle portions.

A roughness and/or fineness of a surface of the inner tube head portion, in particular in the area of the cutting edges, may be at least approximately given by properties of the powder such as, in particular, a granularity or an average grain diameter. Compared to inner tube head portions produced by eroding and possibly subsequent grinding, this results in rougher surfaces with improved cutting properties and an increased service life of the cutting edges and thus of a corresponding inner tube head portion. Furthermore, it has surprisingly been found that appropriately designed cutting edges avoid or prevent obstructions during the removal, in particular suction, of flushing fluid, especially if this carries a large amount of tissue, in particular fibrous tissue. This could be due to the fact that the comparatively rough surface of the cut edge causes cut fibers to be broken and/or shortened to a greater extent.

As part of an inner tube for a surgical cutting device according to the invention, which may be obtainable or manufactured in particular according to the method described above, an inner tube head portion may be formed in the region of a distal end of the inner tube, wherein a cutting window is provided on the inner tube head portion, which has a distal and a proximal end, wherein
a. two opposing cutting edges are provided between the distal and the proximal end of the cutting window, which in particular delimit the cutting window laterally and/or in the circumferential direction,
b. wherein a width of the cutting window initially decreases from the proximal end of the cutting window and/or and increases again towards the distal end.

The width of the cutting window may be regarded as a distance, in particular in the circumferential direction, between the two opposing cutting edges at a position relative to the longitudinal axis, in particular a distance between a first point on a first of the two opposing cutting edges and a second point on a second of the two opposing cutting edges at the same position relative to a coordinate axis running parallel to the longitudinal direction of the inner tube and/or the inner tube head portion.

As part of an inner tube according to the invention for a surgical cutting device with an inner tube head portion at a distal end of the inner tube, which may be obtainable or manufactured in particular according to the method described above, an inner tube head portion may be formed in the region of a distal end of the inner tube, one or more cutting windows being provided on the inner tube head portion, each of which has a distal and a proximal end, and
a. two opposing cutting edges are provided between the distal and the proximal end, in particular which delimit the cutting window laterally and/or in the circumferential direction,
b. wherein the two cutting edges extend from the proximal end initially approximately along a respective helix with a first direction of rotation, and subsequently along a respective helix with a second direction of rotation opposite to the first.

As part of an inner tube according to the invention for a surgical cutting device with an inner tube head portion at a distal end of the inner tube, which may be obtainable or produced in particular according to the method described above, an inner tube head portion may be formed in the region of a distal end of the inner tube, wherein a cutting window, in particular exactly one, is provided on the inner tube head portion, which has a distal and a proximal end, and
a. two opposing cutting edges (2112) are provided between the distal and the proximal end, in particular which bound the cutting window laterally and/or in the circumferential direction,
b. wherein at least one cutting edge (4111, 4112), in particular each of the cutting edges in the direction of the longitudinal axis L, initially has a convex portion 4111a and subsequently, in particular adjoining said convex portion, has a concave portion 4111b or vice versa.

As part of an inner tube for a surgical cutting device according to the invention having an inner tube head portion at a distal end of the inner tube, which may be obtainable or manufactured in particular according to the method described above, the inner tube head portion may have
a. a milling head provided at a distal end,
b. a taper portion remote from the distal end; and
c. a connecting portion provided between the milling head and the taper portion; wherein
d. at least one suction window may be provided on the taper portion, through which liquid may be sucked into the inner tube.

In an interior of the inner tube, in particular in a channel formed in the inner tube, one or more webs, struts or braces may be provided, each of which extends between two regions on an inner side of the inner tube and/or an inner side of a wall forming the inner tube, which regions are offset relative to one another at least with respect to a circumferential direction. The two regions in question may also be offset with respect to the longitudinal axis and/or a direction parallel to the longitudinal axis. At least one of the regions may be provided between the distal and the proximal end of one or more cutting windows with respect to the longitudinal axis and/or a direction parallel to the longitudinal axis. At least one of the regions may be provided between the proximal end of one or more cutting windows and the proximal end of the inner tube with respect to the longitudinal axis and/or a direction parallel to the longitudinal axis. In particular, a first region may be provided between the distal and the proximal end of one or more cutting windows and a second region may be provided along the longitudinal axis between the proximal end of one or more cutting windows and the proximal end of the inner tube.

Such webs, struts or braces increase a mechanical stability of the inner tube, in particular in a region of the inner tube head portion, and effectively counteract undesired bending or twisting as well as other undesired deformations of the inner tube, in particular in the region of the inner tube head portion. This may be particularly important if the inner tube or the inner tube head portion has relatively many and/or relatively long cutting windows, in particular more than two or even more than three cutting windows and/or cutting window lengths with respect to the longitudinal axis and/or a direction parallel to the longitudinal axis of more than 1 cm, 2 cm or even more than 3 cm.

The webs, struts or braces may in particular be formed in one piece with the inner tube and/or the inner tube head portion, and may preferably be created or formed by means of or as part of an additive manufacturing process as described above. Manufacturing by means of material removal, in particular by means of an erosion process, on the other hand, is practically impossible, or at least involves a prohibitively high cost.

Advantageous embodiments of the invention are the subject of the subclaims and/or result from the following description in conjunction with the drawings.

In the following, a preferred embodiment of the invention is described with reference to the accompanying drawings.

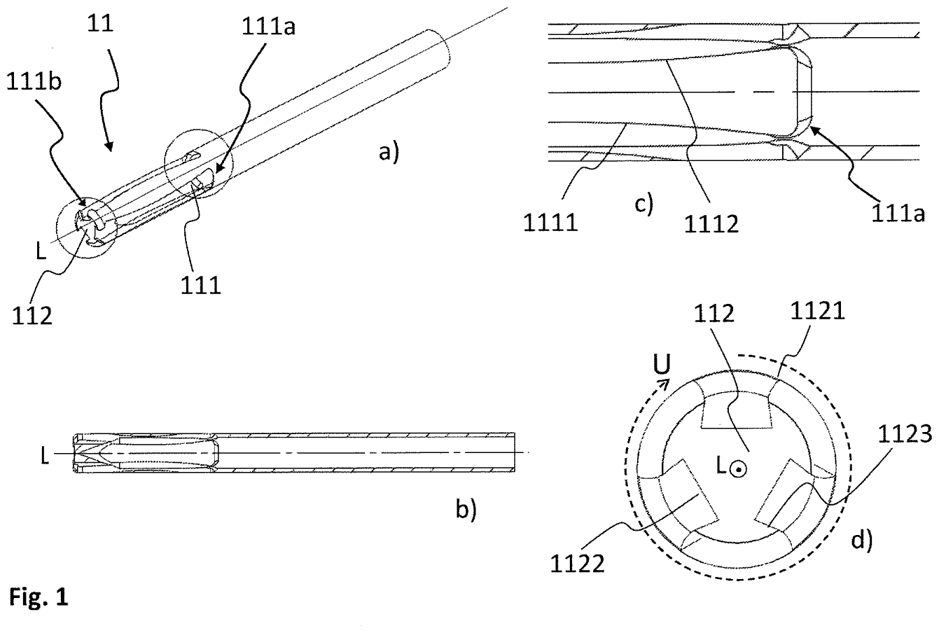

FIG. 1 shows a first embodiment of an inner tube for a surgical cutting device according to the invention;

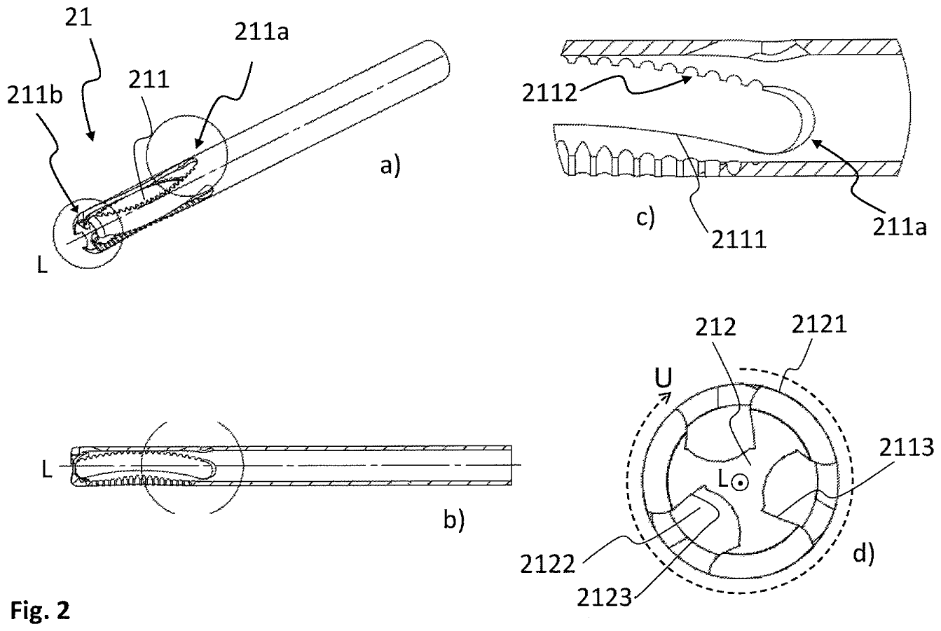

FIG. 2 a perspective view of a second embodiment of an inner tube for a surgical cutting device according to the invention;

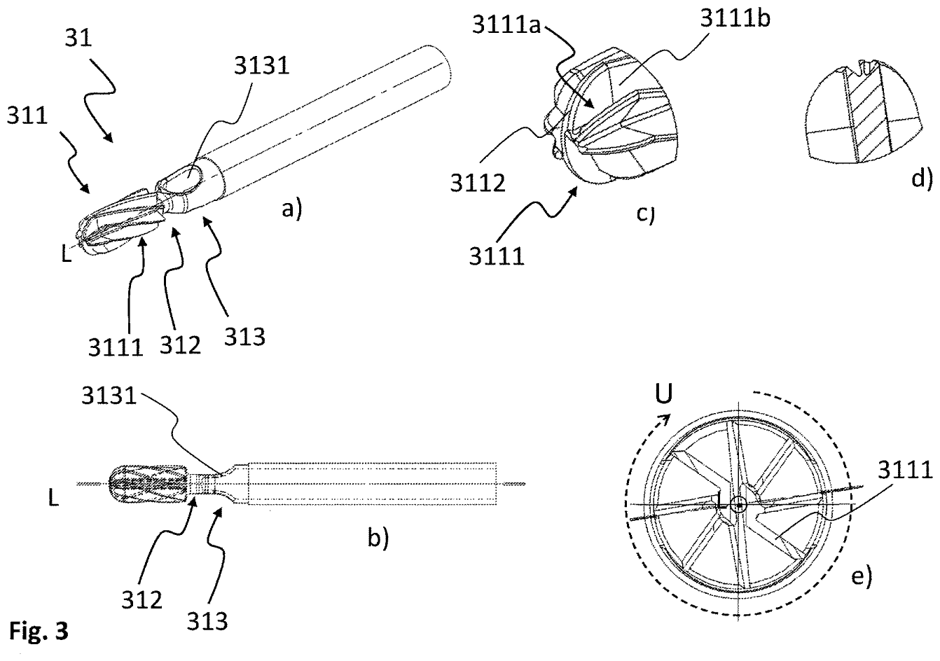

FIG. 3 a perspective view of a third embodiment of an inner tube for a surgical cutting device according to the invention;

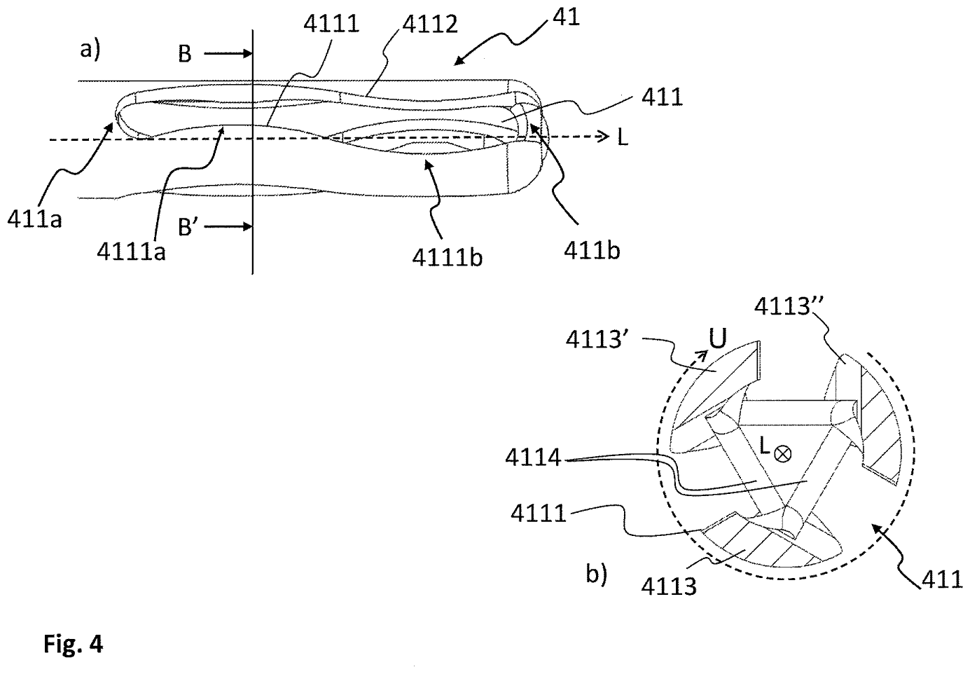

FIG. 4 a perspective view of an inner tube head portion according to a fourth embodiment of an inner tube for a surgical cutting device according to the invention.

### WAYS OF CARRYING OUT THE INVENTION

FIGS. 1a-d show a first embodiment of an inner tube for a surgical cutting device according to the invention, which is obtainable by means of the method described above.

FIG. 1a shows a perspective view of the inner tube according to the invention according to the first embodiment; FIG. 1b a section along a plane containing a longitudinal axis L of the inner tube; FIG. 1c a detail from FIG. 1b; FIG. 1d a top view of the distal end in the direction of the proximal end.

Three cutting windows 111 are formed on an inner tube head portion 11, each of which has a proximal end 111a and a distal end 111b. Between the distal end 111b and the proximal end 111a of each cutting window 111, two opposing cutting edges 1111, 1112 are provided, both of which are smooth or without teeth. The cutting edges delimit the cutting window 111 laterally and/or in the circumferential direction U.

The two cutting edges extend from the proximal end 111a of the, in particular each, cutting window 111 initially towards each other and then away from each other. Accordingly, the distance between the two cutting edges is initially reduced and then increases again. This gives the cutting window 111 a characteristic cushion shape.

A distal end of the inner tube head portion 11 is formed by an inner tube head plate 112, which is oriented perpendicular to the longitudinal axis L of the inner tube head portion and/or the inner tube. The inner tube head plate 112 is designed as a cutting head plate, with three recesses 1122 being provided on a radial edge 1121 of the cutting head plate. Each recess has at least one cutting edge 1123, which in particular is oriented at least approximately perpendicular to the longitudinal axis L and/or in a radial direction of the inner tube head portion 11. With respect to the circumferential direction U, each one of the recesses 1122 is provided at an at least essentially identical position as a respective one of the cutting windows 111, in particular a distal end 111b of the respective cutting window 111.

The recess 1122 and the respective cutting window thus merge into one another in the region of the distal end of the inner tube head portion 11 or of the inner tube.

FIGS. 2a-d show a second embodiment of an inner tube for a surgical cutting device according to the invention, which is obtainable by means of the method described above.

FIG. 2a shows a perspective view of the inner tube according to the second embodiment; FIG. 2b a section along a plane containing a longitudinal axis L of the inner tube; FIG. 2c a detail from FIG. 2b; FIG. 2d a top view of the distal end in the direction of the proximal end.

Three cutting windows 211 are formed on an inner tube head portion 21, each of which has a proximal end 211a and a distal end 211b. Two opposing cutting edges are provided between the distal end 211b and the proximal end 211a of each cutting window 211, of which a first cutting edge 2111 is designed without teeth; straight, flattened teeth with rounded spaces or gaps between the teeth are provided on a second cutting edge 2112. The cutting edges delimit the cutting window 211 laterally and/or in the circumferential direction U.

The two cutting edges extend from the proximal end 211a of the, in particular each, cutting window 211 initially approximately along a respective, in particular first, helix with a first direction of rotation, and subsequently along a respective, in particular second, helix with a second direction of rotation opposite to the first. This gives the cutting window 211 a characteristic boomerang, kidney and/or banana shape.

A distal end of the inner tube head portion 21 is formed by an inner tube head plate 212, which is oriented perpendicular to the longitudinal axis L of the inner tube head portion and/or the inner tube. The inner tube head plate 212 is designed as a cutting head plate, wherein three recesses 2122 are provided on a radial edge 2121 of the cutting head plate. Each recess has a cutting edge 2123, which in particular is oriented at least approximately perpendicular to the longitudinal axis L. With respect to the circumferential direction U, each one of the recesses 2122 is provided at an at least essentially identical position as a respective one of the cutting windows 211, in particular a distal end 211b of the respective cutting window 211. The recess 2122 and the respective cutting window thus merge into one another in the region of the distal end of the inner tube head portion 21 or of the inner tube.

FIGS. 3a-e show a third embodiment of an inner tube for a surgical cutting device according to the invention, which is obtainable by means of the method described above.

FIG. 3a shows a perspective view of the inner tube according to the invention according to the third embodiment with an inner tube head portion 31 at a distal end of the inner tube, which inner tube head portion comprises a milling head 311 provided at a distal end, a tapered portion 313 remote from the distal end; and a connecting portion 312 provided between the milling head 311 and the tapered portion 313. Two opposing suction windows 3131 are provided on the tapered portion 313, through which liquid, in particular rinsing liquid, may be sucked into the inner tube.

The milling head has 8 cutting lamellae 3111. A cutting edge 3112 is formed along a radially outer edge of each cutting lamella. A distal end 3111a of each cutting lamella 3111 extends in a first plane, within which the longitudinal axis L extends. A proximal end 3111b of each cutting lamella 3111 extends in a second plane, which forms an acute angle with the longitudinal axis L, which is preferably less than 30°, in particular at least approximately 20°.

In the tapered area, the inner and outer diameters of the inner tube are reduced, in particular to 30% to 75% of corresponding values of the inner tube middle portion, in particular to at least approximately 50%. The suction windows 3131 are formed by openings in this area, and are thus inclined obliquely forwards. This enables particularly efficient extraction of material removed by the milling head 311.

FIGS. 4a) and b) show an inner tube head portion 41 as may be provided in connection with a fourth embodiment of an inner tube for a surgical cutting device according to the invention, and which inner tube and/or inner tube head portion is obtainable by means of the method described above. Three cutting windows 411 are formed on the inner tube head portion 41, each of which has a proximal end 411a and a distal end 411b. Between the distal end 411b and the proximal end 411a of each cutting window 411, two opposing cutting edges 4111, 4112 are provided, both of which are smooth or without teeth. The cutting edges delimit the cutting window 411 laterally and/or in the circumferential direction U. A respective wall portion 4113, 4113′ or 4113″ is formed between two adjacent cutting windows 411, wherein in particular a cutting window 411 is formed between each two of the wall portions 4113, 4113′, 4113″, and furthermore in particular a respective cutting edge 4111 and 4112 is formed on lateral edges of each wall portion 4113, 4113′, 4113″.

Each cutting edge 4111 and 4112 initially has a convex portion 4111a in the direction of the longitudinal axis L and subsequently, in particular adjacent to said convex portion, a concave portion 4111b or vice versa. Such a design of the cutting edges 4111, 4112 ensures that, regardless of the direction of rotation of the inner tube, the concave and convex portions of the cutting edge cut off tissue, bone and/or other body components at least approximately simultaneously, thereby enabling a more even and straighter cutting action and/or reducing the risk of jamming or blocking.

As can be seen in FIG. 4b), a strut 4114 is formed between each two of the wall portions 4113, 4113′, 4113″. Such struts increase the mechanical stability of the inner tube, in particular of the inner tube head portion.

Although the invention is illustrated and described in detail by means of the figures and the accompanying description, this illustration and this detailed description are to be understood as illustrative and exemplary and not necessarily as limiting the invention. In order not to unnecessarily complicate the understanding of the invention, in certain cases well-known features, structures and/or techniques may not be shown or described in detail. It is understood that those skilled in the art may make changes and modifications without departing from the scope of the following claims. In particular, the present invention covers further embodiments with any combinations of features that may differ from the explicitly described combinations of features.

The present disclosure also covers embodiments with any combination of features mentioned or shown above or below for various embodiments. It also includes individual features in the figures, even if they are shown there in connection with other features and/or are not mentioned above or below. The alternatives of embodiments described in the figures and the description and individual alternatives of their features may also be excluded from the subject matter of the invention or from the disclosed subject matter. The disclosure comprises embodiments which exclusively comprise the features described in the claims or in the embodiment examples as well as embodiments which comprise additional other features.

Furthermore, the term “comprising” and or derivatives thereof does not exclude other elements or steps. Likewise, the indefinite article “a” or “one” and derivatives thereof does not exclude a plurality. The functions of several features listed in the claims may be fulfilled by one unit or one step. The terms “substantially”, “about”, “approximately”, “approximately” and the like in connection with a property or a value also define in particular precisely the property or precisely the value. The terms “about”, “approximately”, “approximately”, “about” etc. in connection with a given numerical value or range may refer to a value or range which is within 20%, within 10%, within 5% or within 2% of the given value or range. All reference signs in the claims are not to be understood as limiting the scope of the claims. An indication that a≈b applies may be understood to mean that |a−b\/(|a|+|b|)<0.2, preferably |a−b)/(|a|+|b|)<0.05, most preferably |a−b|/(|a|+|b|)<0.01 applies, wherein a and b may represent any variables or quantities defined and/or described anywhere in this document or otherwise known to the person skilled in the art. An indication “few” may in particular mean at least two, preferably at least three and/or at most five. An indication “some” may in particular mean at least three, preferably at least five and/or a maximum of nine.

The fact that a feature or a property, for example a specific, in particular geometric, shape, is at least approximately formed, provided or present may mean in particular that manufacturing specifications exist which provide for a specification according to which the feature is formed accordingly, whereby a deviation from the specification can result within the framework of usual manufacturing tolerances known to the person skilled in the art.

Features, properties, etc. placed in brackets in the description may be present and/or required, but do not have to be, and are generally to be regarded as optional. Such features can serve to improve the understanding of the invention by hiding implicit aspects that are not essential to the invention. Such features, properties, etc. can be advantageous.

That an element or feature is extended in one direction or extends in one direction may in particular mean that dimensions of the element or feature are larger in this direction than in other, in particular all other directions, in particular orthogonal directions.

The terms “top”, “bottom”, “upper side”, “lower side” can be understood to mean that a vertical direction V of the kitchen aid shown in the figures runs from top to bottom.

1. A method of manufacturing an inner tube for a surgical cutting instrument comprising
a. an outer tube having an outer tube head portion at a distal end of the outer tube;
b. said outer tube head portion having an outer tube window; wherein
c. the inner tube can be rotatably disposed within the outer tube;
d. an inner tube head portion (11, 21, 31) is provided at a distal end of the inner tube; wherein
e. at least one cutting edge (1112, 2112, 3112) is formed as part of the inner tube head portion which cutting edge is moved out of an interior of the outer tube through the outer tube window upon rotation of the inner tube and is moved back into the interior of the outer tube through the outer tube window upon further rotation; the method comprising the steps of:
f. providing an inner tube middle portion having a distal end;
g. forming the inner tube head portion at the distal end of the inner tube middle portion by means of an additive manufacturing process to obtain the inner tube.

2. A method of manufacturing a surgical cutting instrument comprising
a. an outer tube with an outer tube head portion at a distal end of the outer tube;
b. an inner tube rotatably disposed within the outer tube with an inner tube head portion (11, 21, 31) at a distal end of the inner tube; wherein
c. the outer tube head portion has an outer tube window;
d. at least one cutting edge (1112, 2112, 3112) is formed as part of the inner tube head portion which cutting edge is moved out of an interior of the outer tube through the outer tube window upon rotation of the inner tube and is moved back into the interior of the outer tube through the outer tube window upon further rotation; the method comprising the steps of:
e. providing an inner tube middle portion having a distal end;
f. forming the inner tube head portion at the distal end of the inner tube middle portion by means of an additive manufacturing process to obtain the inner tube;
g. inserting the inner tube into the outer tube.

3. The method according to claim 1 or 2, further comprising:
a. Providing an outer tube middle portion having a distal end;
b. forming the outer tube head portion at the distal end of the outer tube middle portion by an additive manufacturing process to obtain the outer tube.

4. The method according to claim 1, 2 or 3, wherein
a. the inner tube middle portion and/or the outer tube middle portion is formed of stainless steel,
b. the inner tube head portion and/or the outer tube head portion are formed from stainless steel, in particular by means of selective laser melting, SLM, laser cusing or direct metal laser sintering, DMLS.

5. The method according to one of the preceding claims, characterized in that the inner tube and/or the outer tube are formed from stainless steel 1.4443.

6. The method according to one of the preceding claims, characterized in that one or more cutting windows, each with one or more cutting edges, are formed on the inner tube head portion.

7. An inner tube for a surgical cutting instrument with an inner tube head portion (11) at a distal end of the inner tube, wherein a cutting window (111), in particular exactly one, is provided on the inner tube head portion, which has a distal and a proximal end, and
a. two opposing cutting edges (1111, 1112) are provided between the distal and the proximal end, which in particular delimit the cutting window laterally and/or in the circumferential direction,
b. wherein a width of the cutting window, in particular a distance between the cutting edges in the circumferential direction, initially decreases from the proximal end and increases again towards the distal end.

8. The inner tube according to claim 7, wherein at least one or more further cutting windows (111) are provided on the inner tube head portion, each of which has a distal and a proximal end, wherein
a. two opposing cutting edges (1112) are provided between the distal and proximal ends, which in particular delimit the further cutting window or windows laterally and/or in the circumferential direction,
b. wherein a width of the further cutting window or windows, in particular a distance between the cutting edges in the circumferential direction, initially decreases from the proximal end and increases again towards the distal end.

9. An inner tube for a surgical cutting instrument with an inner tube head portion (21) at a distal end of the inner tube, wherein a cutting window (211), in particular exactly one, is provided on the inner tube head portion, which has a distal and a proximal end, and
a. two opposing cutting edges (2111, 2112) are provided between the distal and the proximal end, in particular which bound the cutting window laterally and/or in the circumferential direction,
b. wherein the two cutting edges extend from the proximal end initially at least approximately along a respective first helix with a first direction of rotation, and subsequently at least along a respective second helix with a second direction of rotation opposite to the first direction of rotation.

10. The inner tube according to claim 9, wherein at least one or more further cutting windows (211) are provided on the inner tube head portion (21), each having a distal and a proximal end, wherein
a. two opposing cutting edges (2111, 2112) are provided between the distal and the proximal end, in particular which bound the cutting window laterally and/or in the circumferential direction,
b. wherein the two respective cutting edges extend from the proximal end initially approximately along a respective helix with a first direction of rotation, and subsequently along a respective helix with a second direction of rotation opposite to the first.

11. An inner tube for a surgical cutting instrument comprising an inner tube head portion (41) at a distal end of the inner tube, wherein one or more cutting windows (411) are provided on the inner tube head portion, each of which has a distal and a proximal end, and
a. two opposing cutting edges (2112) are provided between the distal and the proximal end, in particular which delimit the cutting window laterally and/or in the circumferential direction,
b. wherein at least one cutting edge (4111, 4112), in particular each of the cutting edges in the direction of the longitudinal axis L, initially has a convex section (4111a) and subsequently, in particular adjoining said convex section, has a concave section (4111b) or vice versa.

12. The inner tube according to claim 8, 10 or 11, wherein a total of exactly three cutting windows (111, 211, 411) are provided, which are in particular each offset by 120° relative to one another in a circumferential direction.

13. The inner tube according to one of claims 7 to 12, wherein in each case a first cutting edge of the two opposing cutting edges of a cutting window (111, 211, 411) has teeth and a second cutting edge of the two opposing cutting edges of the corresponding cutting window is formed without teeth, in particular smooth.

14. The inner tube according to any one of claims 7 to 13, wherein a distal end of the inner tube head portion (11, 21, 41) is formed by an inner tube head plate (112, 212) which is preferably oriented perpendicular to a longitudinal axis of the inner tube head portion.

15. The inner tube according to claim 14, wherein the inner tube head plate (112, 212) is designed as a cutting head plate, which is characterized in that
a. a recess (1122, 2122) is provided at a radial edge (1121, 2121) of the cutting head plate,
b. the recess has at least one cutting edge (1123, 2113), which in particular is oriented at least approximately perpendicular to the longitudinal axis, and wherein
c. the recess and one of the cutting windows (111, 211) merge into one another.

16. The inner tube according to claim 14, wherein the inner tube head plate (112, 212) is designed as a cutting head plate, which is characterized in that
a. one recess per cutting window is provided on a radial edge (1121, 2121) of the cutting head plate,
b. each recess has at least one cutting edge, which in particular is oriented at least approximately perpendicular to the longitudinal axis, and wherein
c. each one of the recesses merges into a respective one of the cutting windows.

17. An inner tube for a surgical cutting instrument with an inner tube head portion (31) at a distal end of the inner tube, wherein the inner tube head portion comprises
a. a milling head (311) provided at a distal end,
b. a taper portion (313) remote from the distal end; and
c. a connecting portion (312) provided between the milling head and the taper portion; and wherein
d. at least one suction window (3131) is provided on the taper portion, through which liquid may be sucked into the inner tube.

18. The inner tube according to claim 17, characterized in that the milling head (311) comprises a plurality of cutting lamellae, in particular 8 cutting lamellae, a cutting edge being formed along a radially outer edge of each cutting lamella.

19. The inner tube (11, 21, 31, 41) according to one of claims 7 to 18, characterized in that one or more webs, struts or braces (4114) are provided in an interior of the inner tube, in particular in a channel formed in the inner tube, each of which extends between two regions, which are offset relative to one another at least with respect to a circumferential direction, on an inner side of the inner tube and/or an inner side of a wall forming the inner tube.

20. The method according to any one of claims 1 to 6, wherein an inner tube according to any one of claims 7 to 19 is obtained by means of the additive manufacturing method.

21. The method or the inner tube according to one of the preceding claims, wherein the inner tube and/or the outer tube have a diameter d in the range of a few millimeters, in particular with 2.5 mm<d<10 mm, and/or a length l in a range of 15 cm<l<30 cm.
